(19) United States
(12) Patent Application Publication
(10) Pub. No.: US 2012/0009231 A1
(43) Pub. Date: Jan. 12, 2012

(54) APPARATUS AND METHOD FOR DEPOSITION OF FUNCTIONAL COATINGS

(76) Inventors: Anthony Herbert, Cork (IE); Justyna Jaroszynska-Wolinska, Nadbystrzycka (PL); Liam O'Neill, Co. Cork (IE)

(21) Appl. No.: 13/257,072

(22) PCT Filed: Mar. 18, 2010
(86) PCT No.: PCT/EP10/01703

§ 371 Date: Sep. 16, 2011

(30) Foreign Application Priority Data
Mar. 19, 2009 (IE) 2009/0213

(51) Int. Cl. C23C 16/50; C23C 16/455; A61F 2/04; A61K 9/00; C12M 1/00; B32B 9/02; C23C 16/56; B32B 1/08

(52) U.S. Cl. 424/400; 427/569; 427/535; 427/553; 427/551; 118/723 E; 428/36.91; 435/287.1; 435/288.3; 428/532; 428/704

(57) ABSTRACT

A method for deposition of functional coatings comprises igniting a non-thermal equilibrium plasma within an ambient pressure plasma chamber having a gas supply inlet and a plasma outlet; and providing a substrate to be coated adjacent to the plasma outlet. A gas phase pre-cursor monomer is provided to the plasma chamber through the gas inlet. A specific energy is coupled into the plasma during the flow of the pre-cursor through the chamber sufficient to disassociate at least the weakest intra-molecular bond required to allow polymerisation of the pre-cursor when deposited on a surface of the substrate adjacent the plasma outlet, the coupled specific energy not exceeding a specific energy required break intra-molecular bonds required for the functionality of the monomer molecule.

### RELATED APPLICATIONS

This application claims the benefit of priority under 35 U.S.C. §365(c) to International Application PCT/EP2010/001703, filed Mar. 18, 2010, which claims priority to IE2009/0213, filed Mar. 19, 2009, both incorporated herein by reference in entirety.

### FIELD OF THE INVENTION

The present invention relates to an apparatus and method for deposition of functional coatings.

### BACKGROUND

In general there are two plasma types, namely thermal equilibrium and non-isothermal equilibrium plasmas. Thermal equilibrium plasmas are typically hot with temperatures ˜10,000 K and are used in industry as plasma torches, jets and arcs for welding, metallurgy, spray coating, etc.

Non-isothermal plasmas are generally cool and can be employed in manufacturing processes including surface cleaning (removal of unwanted contaminants), etching (removal of bulk substrate material), activation (changing surface energies) and deposition of functional thin film coatings onto surfaces. They used in a multiplicity of industry segments from microelectronics to medical.

Non-isothermal plasmas can be used to deposit, at low temperatures, functional coatings, which conform and adhere well to a substrate surface. The process leaves the bulk of the substrate unchanged. Such coatings allow the surface to have a different set of properties from those of the bulk material of the substrate and, thus, allow the bulk material to have one set of characteristics, e.g. rigidity, while surface may have another independent set of characteristics, e.g. low friction.

Non-isothermal equilibrium plasma polymerization is known in the field of surface functionalization and has applications in diverse areas such as biotechnology, adhesion, electronics and textiles. Plasma polymerization was initially developed under vacuum conditions and used low pressure plasma technology to polymerise gas vapours and produce polymeric coatings in a technique referred to as plasma enhanced chemical vapour deposition (PECVD). In these early systems, the vapour phase precursors were bombarded with aggressive plasma species which produced fragmentation and re-arrangement of the precursor monomers. As a result, a wide variety of random fragments were created which could deposit on to a substrate to produce a thin film layer which contained many of the atoms present in the starting monomer. Although PECVD became well established, the coating functionality remained limited to simple materials such as SiOₓ, SiN or TiO₂ and complex chemistry could not be deposited using such systems.

The term “soft plasma polymerization” (SPP) relates to the ability to plasma deposit a solid film with a very high degree of structural retention of a starting precursor so that the deposited coating retains the molecular complexity, functionality and value of the monomer. An SPP process should avoid fragmentation of the precursor but, at the same time, deliver a cured coating.

Until approximately 1990, plasma polymerization processes were generally regarded as processes in which small molecules could be polymerized to produce thin films with an unspecified chemical structure, consisting predominantly of carbon, hydrogen, fluorine and oxygen- and nitrogen-based functional groups depending on the chemistry of the monomer.

In the last 15 years or so, however, a range of plasma types and process control parameters has been identified for delivering SPP in varying degrees. Thus, control of substrate temperature such as disclosed by G. Lopez and B. D. Ratner, ACS Polym. Mater. Sci. Eng., 1990, 62, 14; reactant pressure and flow rate, absorbed continuous wave power, such as disclosed by V. Krishnamurthy, I. L. Kamel and Y. Wei, J. Polym. Sci.: Part A, Polym. Chem., 1989, 27, 1211; and location of substrates at varying distance from the plasma region, such as disclosed by H. Yasuda, J. Polym. Sci., Macromol. Rev., 1981, 16, 199 have all been used to bring greater levels of control to the polymerization process.

Additionally, pulsed vacuum PECVD systems allow the power coupled to the plasma to be pulsed in a manner that still creates the active species in the plasma, but does not contain enough energy to fragment all of the bonds within a monomer. The resulting active species interacted with gas phase monomers and produced a soft polymerization reaction which deposits coatings with complex functional chemistry, see M. E. Ryan, A. M. Hynes, J. P. S. Badyal, Chem. Mater., 1996, 8, 37-42; and S. Schiller, J. Hu, A. T. A. Jenkins, R. B. Timmons, F. S. Sanchez-Estrada, W. Knoll, R. Forch, Chem. Mater., 2002, 14, 235. Despite the excellent film control offered by this process, these systems are still limited to vacuum processing and this has hindered commercial exploitation of the technology.

A particular form of plasma that has been investigated for surface coating is a pin-to-plane corona, FIG. 1. Pin-to-plane refers to the electrode configuration used to generate the plasma, as opposed to, for example, a wire-to-plate or two opposing parallel plates configurations, while the term corona describes the plasma type.

A corona discharge is a non-arcing, non-uniform plasma discharge which appears as a luminous glow localized in space around a point tip or wire electrode under high applied voltage. The discharge can be filamentary or more homogeneous depending upon the polarity of the electrode.

The true corona is generated in the strong electric fields near sharp points or fine wires. The visible portion of the true corona occurs in the region within the critical radius, at which the electric field is equal to the breakdown electric field of the surrounding gas. The true corona does not occur between two parallel smooth plates, nor in the presence of an insulating coating over the conductor giving rise to it.

The true corona should be distinguished from the plasma type generated by what are loosely called industrial “corona treaters”. Such systems do not have the electrode geometry needed to generate true coronas and, generally, have at least one electrode coated with dielectric. These systems generate a different plasma type known as a dielectric barrier discharge (DBD), so that there is often confusion between the true corona and a dielectric barrier discharge.

The pin-to-plane electrode corona generation configuration can be reduced by removal of the plane electrode to create a single pin electrode system, depending upon correct configuration of other system variables. This single electrode system sees the surrounding ambient as the counter-electrode and will discharge freely from the point of the pin or the thin wire into the surrounding ambient without the need for a solid counter-electrode. In the present specification, this is referred to as “pin corona”. The absence of a solid counter-electrode has advantages in simplification of the equipment configuration and the ability to treat surfaces without regard to their geometry in the z-direction, i.e. along the main axis of the pin or needle.

Pin coronas have not been seen as viable vehicles for deposition of functional coatings at least partly because they are intrinsically small area and highly spatially inhomogeneous and so would tend to deliver small area coatings comprising films of greatly varying thickness and, possibly, chemical composition, across substrate surfaces.

In “HF plasma pencil—new source for plasma surface processing”, J. Janca, M. Klima, P. Slavicek and L. Zajickova, Surface and Coatings Technology, 116-119, (1999), 547-551, an atmospheric pressure 13.56 MHz RF pin corona discharge from a needle electrode is used to deposit unspecified “stable and crosslinked” polymers from siloxanes and cyclofluorbutane in helium or argon, although the process appears to have been conducted through some liquid medium.

In “The Torch Discharge Plasma Source for the Surface Treatment Technology”, V. Kapicka et al, Proceedings of Hakone VII International Symposium on High Pressure, Low Temperature Plasma Chemistry, Greifswald, Germany, 10-13 Sep. 2000, 506-508, the same group used the same system but with no liquid medium to put down hard, low molecular weight CH polymer films from N-hexane vapour. Issues regarding high operational temperature, plasma dimensions and ability to achieve SPP were not fully or at all addressed.

Separately, L. O'Neill et al, “Plasma Polymerised Primers—Improved Adhesion through Polymer Coatings”, Society of Vacuum Coaters, 50^{th} Annual Technical Conference Proceedings, 2007 disclose a pin corona configuration corresponding to the “PlasmaStream” system from Dow Corning Corporation to deposit functional coatings under the brand name “APPLD” (Atmospheric Pressure Plasma Liquid Deposition), see L. -A. O'Hare, L. O'Neill, A. J. Goodwin, Surf. Interface. Anal., 2006, 38 (11), 1519; and J. D. Albaugh, C. O'Sullivan, L. O'Neill, Surf. Coat. Technol., 2008, 203, 844-847.

Other material relating to this work includes: B. Twomey, D. Dowling, L. O'Neill and L -A O'Hare, Plasma Process. and Polym., 2007, 4, S450-454; P. Heyse, R. Dams, S. Paulussen, K. Houthoofd, K. Janssen, P. A. Jacobs, B. F. Sels, Plasma Process. Polym., 2007, 2, 145; and M. Tatoulian, F. Arefi-Khonsari, Plasma Process. Polym., 2007, 4, 360.

However, this system incorporated a nebuliser to inject the monomer precursor into the plasma region in the liquid state as atomized droplets. The introduction of the liquid as an aerosol was thought to protect the bulk of the liquid precursor from the aggressive plasma species by encapsulating it within a droplet of several microns in diameter, thereby minimising fragmentation of the precursor monomers.

The systems of Janca and Dow Corning have either used or been applied through liquids. In the case of Dow Corning, liquid state precursors in the form of atomized droplets have been seen as central to delivery of SPP and target processes, see A. Hynes et al, “Generation and Control of Wide-area Homogeneous Atmospheric Pressure Glow Discharges for Industrial Coating Applications”, Hakone IX International Symposium on High Pressure, Low Temperature Plasma Chemistry, Padova, Italy, 2004. However, there are disadvantages in the use of precursor in the liquid state. The use of aerosol delivery systems produces a number of complexities related to the stability of the spray, control of droplet size, generation of an even precursor distribution over wide areas, the requirement to accurately dispense low volumes of liquid at a constant rate and rapid build-up of unwanted deposits on reactor surfaces.

It is an object of the present invention to mitigate the problems of this prior art.

### SUMMARY OF THE INVENTION

According to the present invention there is provided a method for deposition of functional coatings comprising:

igniting a non-thermal equilibrium plasma within an ambient pressure plasma chamber having a gas supply inlet and a plasma outlet;

providing a substrate to be coated adjacent to said plasma outlet;

providing a gas phase pre-cursor monomer to the plasma chamber through the gas inlet; and

coupling a specific energy into said plasma during the flow of said pre-cursor through said chamber sufficient to disassociate at least the weakest intra-molecular bond required to allow polymerisation of said pre-cursor when deposited on a surface of said substrate adjacent said plasma outlet, said coupled specific energy not exceeding a specific energy required break intra-molecular bonds required for the functionality of the monomer molecule.

Preferably, said plasma comprises a pin corona plasma.

Preferably, said polymerisation comprises cross-linking said monomers.

Preferably, said plasma operates at approximately room temperature so preventing thermal molecular damage to said pre-cursor.

Preferably, said method provides pumping a carrier gas through a liquid phase monomer, or solution thereof, to vaporise at least a portion of said monomer and providing said vaporised monomer to said plasma chamber.

Preferably, said carrier gas comprises one or more of: helium, argon or nitrogen.

Embodiments of the invention provide soft plasma polymerization from gas state precursor using a cool, atmospheric pressure, highly non-isothermal equilibrium, corona discharge from a single, needle/pin geometry electrode.

Electrical characterisation of the plasma suggests that the retention of chemical functionality is related to the low level of power, specifically the low energy density (J/cm³), coupled into the plasma. It appears that with this type of corona discharge, essentially damage-free polymerization of monomer molecules to deposit a functional coating can be readily achieved by use of precursor in the gas state, so that the use of precursor in the liquid state as nebulised droplets is not required to achieve SPP as has been suggested in for example A. Hynes et al referred to above. This would appear to reduce the need for costly and complex liquid delivery apparatus in many applications using low power corona plasma to achieve functional coatings.

The corona plasma type is particularly suited to delivering low specific energy into a reaction zone and, hence, to provide SPP, even using gas precursors. Although the discharge is not a large area coating source, it is perfectly applicable to substrates <1 m² where sophisticated functionality is required for a surface coating.

The pin corona plasma configuration is further suited to ambient pressure operation. This enables industry migration from vacuum batch to continuous processing. This in turn facilitates much simpler and lower cost equipment designs with reduced maintenance requirements due to the lack of vacuum pumps, seals, etc.

The introduction of precursor as gas/vapour rather than liquid allows for standard PECVD equipment (bubblers, mass flow controllers) to be used to generate an easily controlled, even flux of precursor into a system and onto a substrate avoiding many of the problems of the prior art.

### BRIEF DESCRIPTION OF THE DRAWINGS

Embodiments of the invention will now be described, by way of example, with reference to the accompanying drawings, in which:

FIG. 1 is a schematic diagram of a Pin-to-Plane Corona Plasma Discharge Configuration;

FIG. 2 is a schematic of 2-pin Electrode Head of a Pin Corona Discharge Coating System;

FIG. 3 shows the chemical structure of HDFDA;

FIG. 4 is an FTIR spectrum of HDFDA coating deposited for 180 seconds on an NaCl disk using the apparatus of FIG. 2;

FIG. 5 is an XPS spectrum of HDFDA deposited on a Si wafer for 3 minutes using the apparatus of FIG. 2; and

FIG. 6 shows Vₐₚₚ vs. t (Channel 1) and I_{d} vs. t (Channel 2) Corona Discharge characteristics for the apparatus of FIG. 2.

### DESCRIPTION OF THE PREFERRED EMBODIMENTS

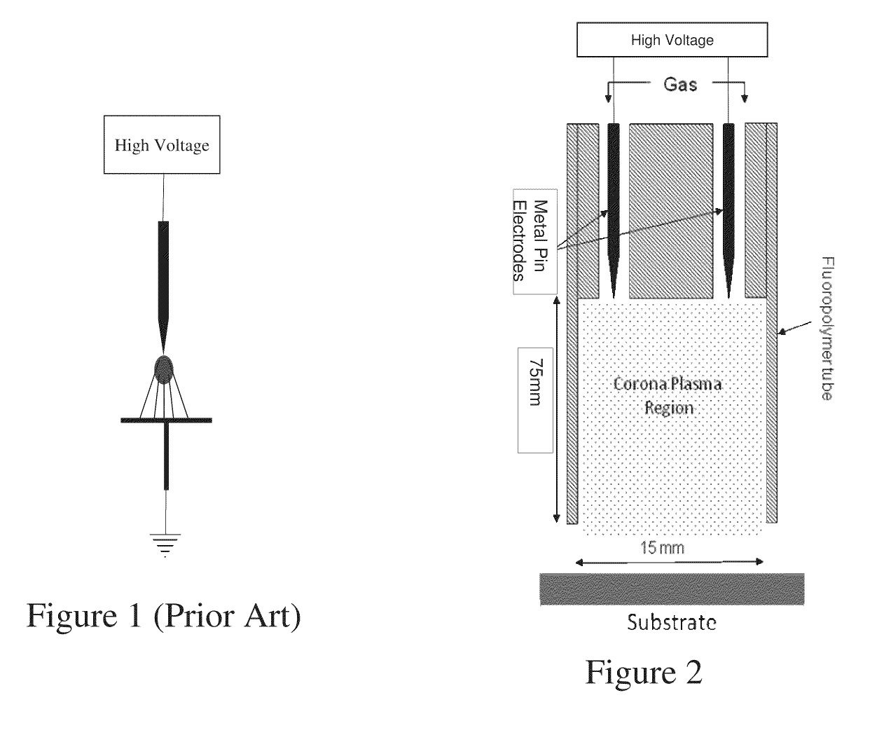

The invention uses a pin corona plasma at atmospheric pressure to achieve soft polymerization with gas state precursors. The electrode can comprise a single sharp pin as shown in FIG. 1 or two or more pins. For example, FIG. 2 shows a schematic of a two pin electrode head of a pin corona coating system which could be used for the present invention. The dimensions provided in FIG. 2 are by way of illustration only and can differ depending upon the details of the system and application.

Preferably, although not necessarily, the electrode head comprises a tubular dielectric housing (hatched in FIG. 2) mounting two tungsten needle pointed electrodes to which are applied in parallel an alternating current high voltage to generate the corona discharge from the needle tips. A space around each electrode allows a mixture of carrier gas and precursor vapour to enter the device. The carrier gas can be, in principle, any gas but it has been found that relatively chemically inert gases such as helium, argon or nitrogen provide the best degree of control over the plasma chemistry and, hence, the coating composition and process. The precursor monomer to be polymerized, if already a gas, is introduced into the corona plasma region of FIG. 2 by controlled pre-mixing in a manifold with the carrier gas. In some processes no carrier gas is necessary.

If the precursor begins as a liquid, carrier gas can bubbled through a volume of precursor held at a controlled temperature in a standard bubbler set up. The precursor is, thus, introduced into the corona discharge region as a vapour. By controlling the flow of carrier gas and bubbler temperature, the flow rate of monomer can be controlled including ensuring that the monomer is provided in primarily vapour rather than liquid phase to the plasma chamber.

The dielectric housing improves process control by minimising the presence of unwanted impurities such as ambient air in the reaction volume generally contained within the housing. A substrate to be coated is placed downstream, preferably of the order of millimetres, from the outlet of the tubular housing and either the housing or the substrate can then be moved or rastered/scanned in the horizontal plane to enable complete and uniform coating of the substrate surface. In this regard, relative movement of the head and substrate is programmed to compensate for the otherwise non-uniform coating provided by the pin corona plasma.

The stream of carrier gas and/or precursor gas/vapour is blown into the tubular housing so that the electrodes come in contact with the gas. Due to the high electric field near the electrode sharp points, any gas ionizes to generate a corona plasma and a mixture of electrons, ions, photons, metastables and other excited states, radicals and molecular fragments can be created in the plasma region, the specific microscopic species being controlled by the gas mix, gas flow rate (i.e. residence time in the plasma) and the applied power coupled into the plasma. The mix of microscopic plasma species is blown by the gas flow towards the open face of the tubular housing and the plasma survives for some distance outside the housing, until the oxygen contained in the ambient air quenches the plasma. A substrate placed adjacent to the tube opening or mouth, receives a flux of such species which react to form a deposit or coating conformal with and well adhered to the surface.

It has been found that the coatings put down by this apparatus and method are cured, i.e. solid, and are soft polymerized with minimal fragmentation of the original monomer molecule and exhibit a high retention of monomer functionality.

Embodiments of the invention achieve SPP of monomer precursors due to the inherently low specific energy [J/cm³] of the pin corona discharge coupled into the plasma volume. It is the inherently low specific energy of the pin corona, in contrast to other plasma types, that makes it predisposed to SPP and, thus, a valuable tool for the fabrication of thin film coatings comprising complex, high molecular weight but sensitive molecules.

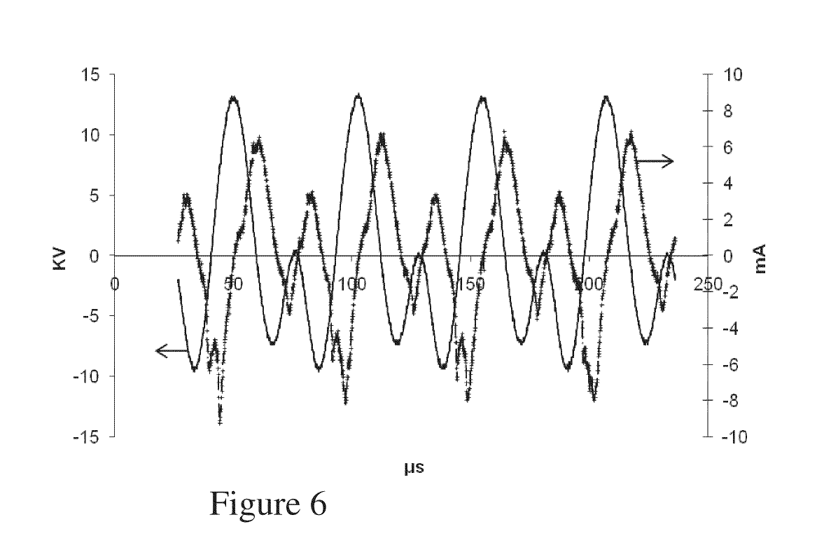

In one embodiment very low frequency electrical power is delivered in parallel to two pins in an electrode head from a modified PTI 100 W power supply from Plasma Technics Inc at a frequency of about 19 kHz and a peak-to-peak voltage of about 23 kV. FIG. 6 shows the Vₐₚₚ vs. time and I_{d} vs. time characteristics of the discharge. It is seen that the peak-to-peak voltage was 23.2 kV and the peak current about 8 mA. The curves show that most of the current is displacement with current about 90 degrees out of phase with voltage. The actual discharge power was calculated as the average over 10 periods of the current-voltage product and was found to be 6.8 W with a +/−6% variation over 5 runs.

Helium-monomer vapour mixtures exited the system through a 75 mm long×15 mm diameter fluoropolymer tubular housing in which the corona plasma was struck. Coatings were deposited onto substrates placed adjacent to the plasma outlet.

The temperature within the plasma was taken at a point 15 mm below the electrodes inside the fluoropolymer tube and within the helium gas flow and any corona discharge. A gas baseline temperature of 8° C. was recorded after 5 minutes of helium gas flow at 14 L/minute in the absence of plasma. Once the plasma was struck and after 10 minutes of discharge, the temperature recorded by the thermometer was found to stabilize at 18° C., clearly indicating the non-thermal equilibrium and low power nature of the discharge.

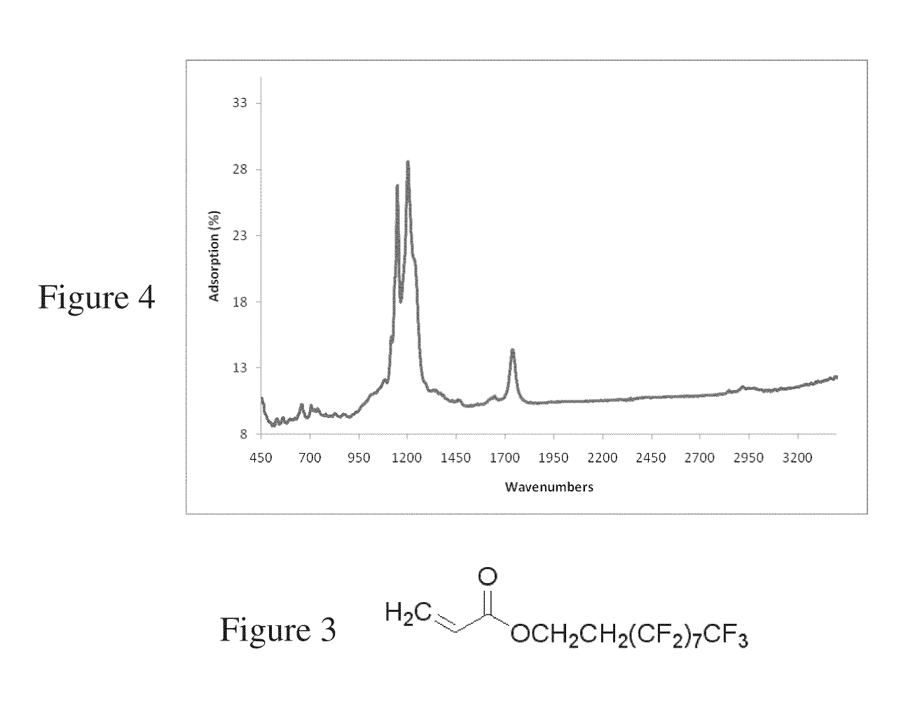

In one process run on this system, 1H, 1H, 2H, 2H-Heptadecafluorodecyl acrylate (HDFDA) was chosen as a precursor monomer as it contains a polymerisable vinyl group and a long perfluoro chain which is easily characterized, FIG. 3. This allows data to be readily compared to prior data published for vacuum polymerisation, see for example, S. R. Coulson, I. S. Woodward, J. P. S. Badyal, S. A. Brewer, C. Willis, Chem. Mater., 2000, 12, 2031; and for aerosol assisted plasma deposition of HDFDA, see L. O'Neill, C. O'Sullivan, “Polymeric Coatings Deposited From an Aerosol-Assisted Non-Thermal Plasma Jet”, Chem Vap. Dep., 2009, 15, 1-6. Furthermore, fluorocarbon films have attracted significant attention as they offer a convenient route to low surface energy coatings which can modify surface properties such as hydrophobicity, oil repellency, cell attachment and chemical inertness.

For electrical characterisation of the system, a Bergoz Instrumentation, France CT-E5.0-B toroidal current transformer with a sensitivity of 5 V/A and 40 mm internal, 72 mm external diameters was used to measure the plasma current (I_{d}); and a North Star PVM-5 high voltage probe with a 1000/1 sensitivity was used to determine the applied voltage (Vₐₚₚ). The Bergoz current transformer toroid was positioned around the fluoropolymer tube of FIG. 2 and 10 mm along the tube from the needle tips to capture the plasma discharge while the high voltage probe was applied at the output of the power supply. The outputs of both probes were captured on a Tektronix TDS 2024 four channel digital storage oscilloscope with a 200 MHz bandwidth.

Fourier Transform Infra-Red (FTIR) data was collected on a Perkin Elmer Spectrum One FTIR. Coatings were deposited directly onto NaCl disks and spectra were collected using 32 scans at 1 cm⁻¹ resolution.

Contact angle measurements were obtained using the sessile drop technique using an OCA 20 video capture apparatus from Dataphysics Instruments. Drop volumes of 1.5 μl were used and images were collected 30 seconds after placing the droplet on the surface. Surface energy was then determined using the OWRK (Owens, Wendt, Rabel and Kaelble) method.

X-ray photoelectron spectroscopy (XPS) was carried out on a VSW spectrometer consisting of an hemispherical analyser and a 3 channeltron detector. All spectra were recorded using an Al Kα X-ray source at 150 W, a pass energy of 100 eV, step size of 0.7 eV, dwell time of 0.1 s with each spectrum representing an average of 30 scans.

Film thickness and thickness profile/mapping of the coatings was determined by a Woollam M2000 variable angle ellipsometer.

HDFDA was introduced into the plasma as a vapour from a standard bubbler set up. By controlling the flow of carrier gas and the bubbler temperature, the flow rate of the monomer could be altered. The bubbler temperature was set to 56° C. and the helium flow to 14 slm. This produced a series of cured dry coatings which were deposited for times of 10, 30 and 180 seconds. Gravimetric measurements indicate an average flow rate of 0.07674 g/min or 126 μL/min of monomer into the device at 56° C.

FTIR analysis was carried out to probe the chemistry of the deposited films and a typical spectrum is shown in FIG. 4. The presence of the dominant peaks centred at 1150 and 1200 cm⁻¹ in the spectra of the coatings correspond to the CF₂ and CF₃ groups of the perfluoro chain. As both fluorocarbon peaks are still well resolved, it can be deduced that the fluorocarbon chain has not undergone significant levels of fragmentation and degradation. Further examination of the main peak at 1205 cm⁻¹ clearly shows a systematic increase in peak intensity with time (Table 1), indicating that thicker coatings are deposited at longer times.

Inspection of the spectra clearly shows loss of the monomer peaks at 1625, 1635, 1412, 1074 and 984 cm⁻¹ corresponding to loss of the C═C bonds of the acrylate group. However, the peak at 1738 cm⁻¹ due to the carbonyl group of the acrylate is still retained in the coating. This indicates that a controlled polymerization of the precursor has occurred through disassociation of the vinyl group of the monomer with retention of the functional chemistry of the larger fluorocarbon chain, as seen in pulsed vacuum and aerosol assisted atmospheric pressure plasma processes referred to above.

Inspection of the region between 2800-3400 cm⁻¹ shows an absence of peaks above 3000 cm⁻¹ which could be associated with the symmetrical and asymmetrical bending and stretching of the C—H bonds of the vinyl group. Two distinct features are detected at 2851 and 2921 cm⁻¹ which are characteristic of the asymmetric and symmetric stretching of saturated CH₂ groups. There is evidence of a weak peak at 2874 cm⁻¹ and a broad peak from 2940-2990 cm⁻¹ which may be due to the symmetric and asymmetric stretch of a terminal methyl group. However, the low signal to noise ratio prevents unambiguous assignment of these features. This loss of vinyl derived peaks, coupled to the presence of saturated alkanes, fluorocarbon and carbonyl signals, further indicates that the plasma reaction is driven through a controlled polymerisation of the vinyl group with conversion to the alkane.

An additional peak can be detected at 1125 cm⁻¹ in the spectra of both these samples and in the spectra of previously published plasma polymerized HDFDA coatings. Although not unambiguously assigned, this could be a secondary C—O species produced due to oxidation of the polymer by the plasma.

Contact angle analysis was carried out to probe the surface energy of the coated substrates. As shown in Table 1, the hexadecane contact angle values were largely independent of deposition time. All samples were found to produce significantly higher hexadecane contact angle values than the uncoated wafer (15°). All coated samples were found to be hydrophobic, with water contact angle values in excess of 90°. The water contact values were found to increase with increased deposition time. This may be explained in terms of increasing surface coverage of the substrate with increased processing time.

TABLE 1
XPS, contact angle and thickness data for HDFDA on Silicon

| | | | | | Contact Angle Analysis | | | FTIR | |
|---|---|---|---|---|---|---|---|---|---|
| Deposition | XPS Elemental | | | | | | Surface | peak | Ellipsometry |
| Time | Composition (%) | | | | Water | Hexadecane | Energy | height | thickness |
| (sec) | Si | C | O | F | (°) | (°) | (mJ/m²) | (a.u.) | (nm) |
| 180 | 0 | 41 | 8 | 51 | 114 | 76 | 11 | 17.52 | — |
| 30 | 2 | 40 | 8 | 50 | 112 | 77 | 11 | 6.28 | 50 |
| 10 | 39 | 20 | 17 | 24 | 97 | 76 | 16 | 1.53 | 10 |

XPS analysis of the coatings was also undertaken to determine their elemental content. XPS analysis of the 10 second sample revealed significant levels of silicon. This suggests that the coating is either patchy or else the coating thickness may be below 10 nm which would result in concurrent analysis of the substrate and coating occurring during the analysis. High levels of oxygen were also detected. These may be derived from oxidation of the coating or from the native silicon oxide present on the wafer surface. The presence of a patchy coating coupled to significant oxidation of the deposit may help to explain the relatively low water contact angle value produced by the 10 second coating.

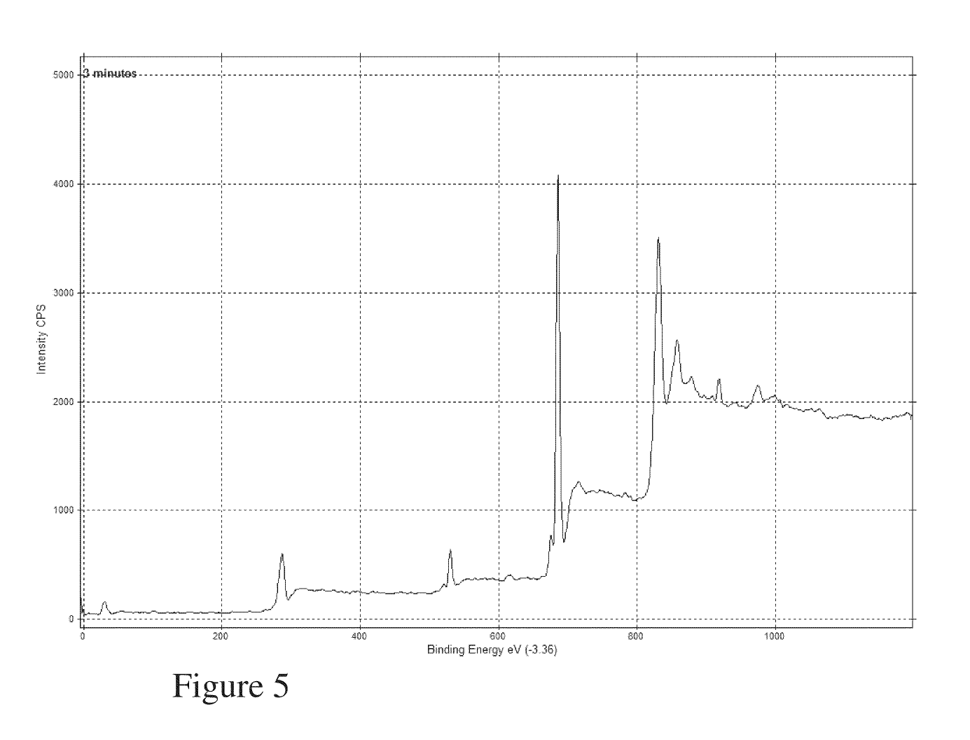

For the coatings deposited at longer times of 30 and 180 seconds, see FIG. 5, the elemental composition of the coating is very similar to that of the un-reacted monomer (41% C, 53% F and 6% O). The spectra from these samples are almost completely devoid of Si, indicating complete coverage of the substrate with a thick polymer layer. A slight increase in oxygen content was detected in the coatings which can be attributed to some minor oxidation of the deposited material by the plasma. However, the results for these two samples are largely similar to results previously seen in soft plasma polymerization reactions and agree with the FTIR data in suggesting that the functionality of the monomer has been largely retained in the coating.

Ellipsometry data was collected from the 10 second and 30 second samples. These coatings were found to have thickness values of 10 and 50 nm respectively, indicating that the deposition rate was in the region of 60-100 nm/min. This is significantly higher than the deposition rates quoted for vacuum plasma coatings produced from HDFDA and is similar to the deposition rates seen in aerosol assisted atmospheric pressure plasma deposition of a range of precursors. Thickness mapping of the coated wafers indicates that the coating occupies a circular region of approximately 3-4 cm in diameter on the wafer surface. Attempts to extract thickness data from the 180 second sample were unsuccessful due to the rough nature of the deposited coating. However, extrapolating coating thickness from the peak heights in the FTIR spectrum would suggest that the 180 second coating is approximately 3 times thicker than the 30 second coating.

Within HDFDA, the dissociation energies of the various bonds are as follows: C—C 348 kJ/mol, C—O 360 kJ/mol, C—H 413 kJ/mol, C—F 488 kJ/mol, O═O 498 kJ/mol and the pi-bond of the C═C bond approximately 264 kJ/mol.

If we attempt to determine the specific energy of the plasma on the following assumptions;
the helium is only an inert background gas and the plasma directly or indirectly, e.g. via helium metastables, eventually imparts all energy to the HDFDA;
such energy is partitioned evenly over all HDFDA molecules; and
the HDFDA gas is, again, modeled as an Ideal Gas at SLC,
   a specific energy of 54 J/cm³ or 1327 kJ/mol or 35 eV/entity would be provided. However, this assumes all of the total discharge energy finds its way into the HDFDA molecules, and this is not thought to be true in practice. For example, substantial discharge energy is likely to be both absorbed by the surfaces contacting the plasma (e.g. through quenching of helium metastables) and lost by radiation before reaching an HDFDA molecule. Furthermore, some proportion of the helium atoms is likely to retain absorbed energy throughout their residence time in the plasma and until and including relaxation back to the ground state without transferring it to HDFDA molecules.

Thus, some part of the specific energy coupled into the plasma never reaches the HDFDA and is not available to drive its polymerization. Such deductions from the specific energy value of 1327 kJ/mol could therefore result in a value not inconsistent with the energy needed to dissociate the C═C pi-bond (˜264 kJ/mol), but which maintains the C—C, C—O, C—H, C—F and O═O bonds.

Film analysis data shows that although the C═C pi-bond is dissociated, the next highest bond dissociation energy, the C—C bond at 348 kJ/mol, is not achieved by the process so that the upper limit of specific energy available for HDFDA fragmentation from this process must be <348 kJ/mol. Thus, this particular plasma type running this process appears to deliver the right specific energy to the plasma region sufficient to break the weakest monomer bond enabling the molecule to react and polymerise but insufficient to break higher energy bonds, in particular those of functional sites. In short, the monomer is not fragmented and the process delivers soft polymerization.

By introducing the fluorocarbon monomer vapour into such a helium corona, it was possible to deposit a cured polymeric coating which retained the chemical structure of the precursor monomer so that the process can be considered to provide soft plasma polymerization (SPP). The coating was hydrophobic and was put down at reasonable deposition rates. Analysis of the coatings clearly shows that the precursor has undergone a controlled polymerization through the vinyl component of the acrylate group with minimal fragmentation of the functional chemistry of the monomer. The resultant coatings produced XPS and FTIR spectra which could previously only be produced by pulsed vacuum plasma or by aerosol assisted plasma processing.

It will be appreciated that apart from the vinyls described above, other bonds that could be disassociated to assist in polymerization include: alkyne, diene, aromatic, acrylate or methacrylate bonds.

In still another example, hexamethyldisiloxane (HMDSO) was deposited using the above-described apparatus. For the process parameters outlined below, the effective specific energy of the plasma is calculated as follows:

Helium flow rate=5 L/min=83.33 cm³/s

Plasma volume in tube 75 mm×15 mm diameter=13.26 cm³

Plasma power=6.8 W
∴ Specific plasma power=0.5129 W/cm³
Residence time in plasma=13.26/83.33 s=0.1591 s
∴ Specific energy of plasma=0.5129×0.1591=0.0816 J/cm³

Of the various bonds within the molecule, Si—CH₃, Si—O, Si—CH₂, and Si—H, the Si—C bond has the lowest dissassociative energy. The above settings provide a specific energy indicated sufficient to break this bond and to provide soft plasma polymerization.

In P. Heyse, R. Dams, S. Paulussen, K. Houthoofd, K. Janssen, P. A. Jacobs, B. F. Sels, Plasma Process. Polym., 2007, 2, 145 referred to above, a non-thermal equilibrium, atmospheric pressure plasma of the dielectric barrier discharge (DBD) type is used with a view to depositing soft polymerized coatings containing bio-molecules such as enzymes using the lowest possible plasma power.

Heyse started with the lowest possible power at which they could successfully generate a from the chosen precursor. In Heyse, plasma and incremented this power until they could get a coating Table 1 column 5, the results for 22 precursors including HMDSO are shown. For HMDSO, a power of 1.20 W/cm² was required.

This can be converted to a specific energy for their HMDSO coating process as follows:

Volume of plasma region=165×180×2 mm³=59.4 cm³

Helium flow rate=20 L/min=333.33 cm³/s

∴ Residence time in plasma=59.4/333.33=0.1782 s
Power density=1.20 W/cm²
∴ Specific power=1.20 (W/cm2)/0.2 (cm)=6.0 W/cm³
∴ Specific energy of plasma=6.0×0.1782=1.0692 J/cm³

From the above calculations, it can be seen that the corona type plasma used in the illustrated example of the present invention has an energy density a factor of ×13 lower than that of the DBD type plasma.

It will also be appreciated that apart from Helium used in the above described examples, other gases including H₂, N₂, Ar, and O₂ or mixtures thereof could be used as carrier gasses depending on the coating to be deposited.

As well as the functional molecules described above it will be appreciated that the invention is equally applicable to the deposition of biologically active coatings onto substrate surfaces. These coatings could include: DNA oligonucleotides, mRNA transcripts including viral plasmids, a functional biologically active protein with an NH₃ terminal, polysaccharide, a catalytic enzyme including arginase, a monoclonal or polyclonal antibody in either complete or Fab fragment form, a hormone including: human chorionic gonadotropin or a steroid, a primary cell, a cell derived from a tumour, a surface receptor, a core receptor, animal or human tissue, a bacterial/viral or pryon microorganism, or human or animal anti-IgG/M to specific protein antigens.

The functional monomers for such coatings typically polymerise through disassociation of a hydroxyl group, a relatively weak bond capable of being disassociated with the level of specific energies disclosed above without damaging the functional remainder of the molecule. Other reactive bonds found within these molecules include thiols, amines and carboxylic acids which can readily participate in plasma polymerisation reactions. Other polymerisable functionalities include cyclic, alicyclic or aromatic rings.

Where biological material does not readily polymerise, it could be encapsulated within polymers formed from an evaporated solvent. For example, active DNA or RNA could be mixed into say HMSO and sprayed into an ante-chamber where the HMDSO evaporates. The vapour could then be introduced into the plasma where a reaction ensues causing the HMSO to polymerise and thereby physically surround and bind the biologically active material to the surface, with minimal chemical reactions involving the biologically active material.

Examples of surfaces which could be coated include stents to treat artery disease, bio-sensors for medical diagnostics, environmental monitoring and industrial process control, assay plates, lab-on-a-chip and biochips, micro-fluidic devices, implanted medical devices with coatings to encourage or inhibit tissue growth, proteomics/genomics, etc.

A feature of virtually all bioactive coatings is that they comprise as the active component large, relatively high molecular weight molecules up to and including proteins, macromolecules (including biopolymers) and living cells. Such molecules are typically difficult to handle, to process and to deposit as a coating without causing damage to or denaturing the molecule and, thus, destroying its functionality and the value of the device or product.

Typically, bio-functional coatings are currently deposited using wet chemical techniques and employ multiple deposition stages. This involves the use of unwanted solvents, binders, linkers and other chemical entities that are expensive, hazardous and not production friendly. Thus, for example, a typical conventional bio-molecule immobilization technique can involve more than 20 wet processing steps using 10 chemicals/solutions and a total process time of hours. Furthermore, such wet processing is inherently isotropic so that patterning of the bio-functional coating to enable new devices or improved performance is generally not possible or only possible with great difficulty. The use of wet processing in the manufacture of devices and products based upon bioactive coatings therefore results in problems for the bio-manufacturing industry including extended processing times, multiple step process complexity, process optimisation, control and reproducibility difficulties, difficulty in patterning of coatings and cost.

Using the method of the present invention, this wet bio-coating can be replaced with a single step, dry process namely plasma depositing bio-active coatings. This can provide better process control with reduced processing time and cost, as well as providing a directional process highly suited to patterning of the bio-coating.

To introduce large, non-volatile bioactive materials into the plasma, the material in question can be dissolved in a highly volatile solvent, sprayed into a heated chamber in which the solvent evaporates and the molecule is then carried into the plasma in a vapour phase. Alternatively, techniques such as electrospray ionisation have been developed to deliver large molecules as charged particles into mass spectrometers and similar techniques can be used to deliver the bioactive molecules in the gas state into the plasma zone.

Additional monomers may also be added to the plasma to provide additional features. Such features may include a requirement such as the formation of a thicker coating, or to increase the cross-linking of the coating. Such features are well known to a person skilled in the art.

In order to further enhance control of the polymerisation process, the pin corona plasma may be pulsed (as in the prior art for low pressure) by repetitive switching on and off of the applied power generating the plasma.

In order to further enhance control of the properties of the functional coating such as adhesion to the substrate, coating densification or degree of cross-linking, additional plasma, ultra-violet, electron beam, ion beam or other energetic processes may be applied to the surface either before or after deposition of the functional coating.

Many variations on the specific embodiments of the invention described will be readily apparent and, accordingly, the invention is not limited to the embodiments hereinbefore described which may be varied in both usage and detail.

1. A method for deposition of functional coatings comprising:
igniting a non-thermal equilibrium plasma within an ambient pressure plasma chamber having a gas supply inlet and a plasma outlet;
providing a substrate to be coated adjacent to said plasma outlet;
providing a gas phase pre-cursor monomer to the plasma chamber through the gas inlet; and
coupling a specific energy into said plasma during the flow of said pre-cursor through said chamber sufficient to disassociate at least the weakest intra-molecular bond required to allow polymerisation of said pre-cursor when deposited on a surface of said substrate adjacent said plasma outlet, said coupled specific energy not exceeding a specific energy required break intra-molecular bonds required for the functionality of the monomer molecule.

2. A method according to claim 1 wherein said plasma comprises a pin corona plasma.

3. A method according to claim 1 wherein said polymerisation comprises cross-linking said monomers.

4. A method according to claim 1 wherein said plasma operates at approximately room temperature so preventing thermal molecular damage to said pre-cursor.

5. A method according to claim 1 further comprising: pumping a carrier gas through a liquid phase monomer to vaporise at least a portion of said monomer and providing said vaporised monomer to said plasma chamber.

6. A method according to claim 5 wherein said monomer is in solution.

7. A method according to claim 5, said carrier gas comprises predominantly one or more of: helium, argon or nitrogen or mixtures thereof.

8. A method according to claim 1 further comprising: dissolving a bio-active material in volatile solvent; and spraying said solution into a heated chamber prior to providing said vaporised solution to said plasma chamber.

9. A method according to claim 1 wherein said monomer includes one or more of: DNA oligonucleotides, mRNA transcripts including viral plasmids, a functional biologically active protein with an NH₃ terminal, polysaccharide, a catalytic enzyme including arginase, a monoclonal or polyclonal antibody in either complete or Fab fragment form, a hormone including: human chorionic gonadotropin or a steroid, a primary cell, a cell derived from a tumour, a surface receptor, a core receptor, animal or human tissue, a bacterial/viral or pryon microorganism, or human or animal anti-IgG/M to specific protein antigens.

10. A method according to claim 1 wherein said weakest intra-molecular bond includes one or more of: a hydroxy, thiol, amine, or carboxylic acid bond.

11. A method according to claim 1 wherein said monomer includes one or more of a:
cyclic, alicyclic or aromatic ring.

12. A method according to claim 1 wherein the monomer includes one of either: HDFDA or HMDSO.

13. A method according to claim 1 wherein the weakest intra-molecular bond includes one of: a vinyl, alkyne, diene, aromatic, acrylate or methacrylate bond.

14. A method according to claim 1 comprising moving said substrate relative to said plasma outlet to compensate for a non-uniformity of coating provided by said method and to provide a required coating of said substrate.

15. A method according to claim 1 comprising pulsing the power applied to said plasma.

16. A method according to claim 1 further comprising applying one or more of: a plasma, ultra-violet radiation, an electron beam or an ion beam to the surface either before or after depositing the functional coating to enhance the properties of the functional coating.

17. A method according to claim 8 wherein said volatile solvent includes a monomer having said weakest intra-molecular bond and wherein said bio-active material is bound within said polymerised monomer when deposited on said substrate surface.

18. An apparatus for deposition of functional coatings comprising:
a plasma chamber incorporating: one or more electrodes, a gas inlet and a plasma outlet exposed to ambient pressure;
an ignition system operatively connected to said electrodes for providing a non-thermal equilibrium plasma within the plasma chamber;
means for providing a substrate to be coated adjacent to said plasma outlet and for moving said substrate relative to said plasma outlet; and
a gas supply in fluid communication with said gas inlet for providing a gas phase pre-cursor monomer to the plasma chamber,
wherein ignition system and said gas supply are controllable to couple a specific energy into said plasma during the flow of said pre-cursor through said chamber sufficient to disassociate at least the weakest intra-molecular bond required to allow polymerisation of said pre-cursor when deposited on a surface of said substrate adjacent said plasma outlet, said coupled specific energy not exceeding a specific energy required break intra-molecular bonds required for the functionality of the monomer molecule.

19. An apparatus as claimed in claim 18 wherein said plasma chamber comprises a dielectric tube in which said electrodes and gas inlet are provided at one end and wherein said plasma outlet is formed at an opposite end.

20. An apparatus as claimed in claim 18 comprising two needle electrodes and wherein said ignition system is arranged to provide power at a frequency in the range 5-100 kHz, and preferably at 19 kHz to said plasma.

21. A substrate coated according to the method of claim 1, said substrate including one of:
a stent, a bio-sensor for medical diagnostics, a sensor for environmental monitoring or industrial process control, an assay plate, a biochip, a micro-fluidic device, a medical device for encouraging or inhibiting tissue growth or proteomics/genomics.
